(19) United States
(12) Patent Application Publication
(10) Pub. No.: US 2004/0033208 A1
(43) Pub. Date: Feb. 19, 2004

(54) Antifungal compositions containing an antibiotic and one or more amidoamines

(76) Inventors: Gerald D. Cagle, Fort Worth, TX (US); Barry A. Schlech, Burleson, TX (US); Joseph W. Hiddene, Arlington, TX (US); G. Michael Wall, Forth Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/432,427

(22) PCT Filed: Apr. 27, 2001
(86) PCT No.: PCT/US01/13559

(30) Foreign Application Priority Data
Dec. 20, 2000 (US) 60257310

(51) Int. Cl. A61K 31/74

(52) U.S. Cl. 424/78.02; 424/78.04; 424/78.07

(57) ABSTRACT

Antimicrobial compositions containing one or more topically active antibiotics (e.g., Natamycin) and one or more amidoamines are described. The amidoamines enhance or supplement the antimicrobial activity of natamycin or other topically active antibiotics. The compositions are particularly useful in treating or preventing fungal infections of the eye, ear, nose and throat, as well as sterilizing these tissues prior to surgery or other medical procedures.

### BACKGROUND OF THE INVENTION

The present invention is directed to improved compositions and therapies for treating or preventing fungal infections. The invention is particularly directed to the topical treatment or prevention of ophthalmic, otic and nasal infections, as well as to the sterilization of these tissues.

There are few effective therapies for treating fungal infections of the eyes, ears, nose, or throat. The antibiotic natamycin is currently utilized to treat ophthalmic fungal infections. A topical ophthalmic composition containing natamycin is marketed under the name NATACYN® (natamycin ophthalmic suspension, USP) 5% Sterile by Alcon Laboratories, Inc., Fort Worth, Tex.

Although the topical ophthalmic use of natamycin has generally proven to be effective in containing fungal infections, there is a need for improved therapies to treat and prevent fungal infections of the eye, ear, nose and throat.

### SUMMARY OF THE INVENTION

The present invention is directed to the use of certain amidoamines to enhance or supplement the antifungal activity of natamycin or other antibiotics. The amidoamines enhance the antifungal activity of natamycin and other antibiotics, but also provide some antibacterial activity. Moreover, the amidoamines are relatively nontoxic to delicate tissues, particularly corneal tissues, but also the mucosal tissues of the ear, nose and throat. This lack of toxicity enables the compositions of the present invention to achieve a significantly higher degree of control of fungal infections without creating the risk of toxicological side effects that might otherwise undermine the overall efficacy of the compositions. The relative mildness of the amidoamines is particularly an advantage in patients whose ophthalmic, otic or nasal tissues have been compromised by means of a surgical procedure, physical injury or infection.

### DESCRIPTION OF PREFERRED EMBODIMENTS

The compositions utilized in the present invention comprise one or more topically active antibiotics, one or more amidoamines, and a pharmaceutically acceptable vehicle for these agents. The compositions are formulated in a manner suitable for topical application to ophthalmic, otic or nasal tissues.

The antibiotics utilized in the present invention may be generally described as being selected from the group consisting of aminoglycosides, quinolones and natamycin. The aminoglycosides and quinolones are both well-known classes of antibiotics. Examples of aminoglycoside antibiotics that may be utilized include tobramycin, gentamicin, framycetin and gramicidin. Examples of quinolone antibiotics that may be utilized include ciprofloxacin, moxifloxacin, ofloxacin, gatifloxacin, levofloxacin, norfloxacin, enterofloxacin and trovafloxacin.

As indicated above, natamycin is also a well-known antibiotic. However, unlike the aminoglycoside and quinolone antibiotics mentioned above, the prior usage of natamycin has been primarily directed to the topical treatment of fungal infections.

The present invention is directed to advancing the state of the art in the field of topical therapies for fungal infections of the eye, ear, nose and throat. Consequently, the use of an antibiotic that is particularly well suited for treating fungal infections is preferred. The use of natamycin in the compositions and methods of the present invention is preferred for this reason. However, the basic principle of the present invention, which is that the amidoamines described herein may be utilized to enhance or supplement the antifungal activity of antibiotics, is also applicable to antibiotics other than natamycin, such as those described above.

The amidoamines utilized in the present invention comprise one or more compounds of the following formula, or pharmaceutically acceptable salts thereof (e.g., hydrohalide salts):

wherein:

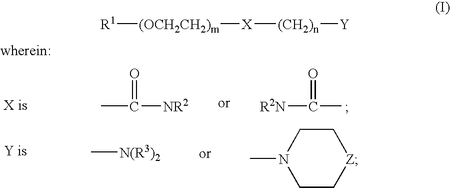

Z is oxygen or NR⁴;

R¹ is C₆-C₁₈ saturated or unsaturated alkyl, alkylaryl, or alkoxyaryl;

m is zero to 16;

n is 2 to 16;

R¹, R¹, and R⁴ are independently hydrogen, C₁-C₈ saturated or unsaturated alkyl or hydroxyalkyl, or a pharmaceutically acceptable salt thereof.

The compounds wherein m is 0 to 5, n is 2 to 4, R² is hydrogen or methyl, R³ is methyl or ethyl, and R⁴ is hydrogen, methyl or hydroxyethyl are particularly preferred, as are the compounds of Table 1:

TABLE 1

| COMPD. | | | | | | | | | |
|---|---|---|---|---|---|---|---|---|---|
| No. | R¹ | M | n | X | R² | Y | R³ | Z | R⁴ |
| 1 | C₁₇ | 0 | 3 | CONR² | H | N(R³)₂ | CH₃ | — | — |
| 2 | C₁₃ | 0 | 2 | CONR² | H | N(R³)₂ | CH₃ | — | — |
| 3 | C₁₃ | 0 | 2 | CONR² | H | N(R³)₂ | C₂H₅ | — | — |
| 4 | C₁₃ | 0 | 3 | CONR² | H | N(R³)₂ | CH₃ | — | — |
| 5 | C₁₁ | 0 | 3 | CONR² | H | N(R³)₂ | CH₃ | — | — |
| 6 | C₁₁ | 0 | 3 | CONR² | H | N(R³)₂ | C₂H₅ | — | — |
| 7 | C₁₁ | 0 | 3 | CONR² | H | 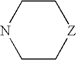 | — | O | — |
| 8 | C₁₄ | 0 | 2 | R²NCO | H | 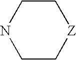 | — | N | H |
| 9 | C₁₃ | 0 | 3 | CONR² | H | 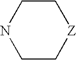 | — | N | CH₃ |
| 10 | C₁₃ | 0 | 3 | CONR² | CH₃ | N(R³)₂ | CH₃ | — | — |
| 11 | C₁₃ | 0 | 3 | CONR² | H | 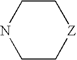 | — | N | C₂H₄OH |
| 12 | C₁₂ | 5 | 3 | CONR² | H | N(R³)₂ | CH₃ | — | — |
| 13 | C₁₂ | 4 | 2 | R²NCO | H | N(R³)₂ | CH₃ | — | — |
| 14 | C₁₂ | 0 | 3 | CONR² | H | N(R³)₂ | CH₃ | — | — |
| 15 | C₁₁ | 0 | 3 | CONR² | CH₃ | N(R³)₂ | CH₃ | — | — |
| 16 | C₁₁ | 0 | 3 | CONR² | H | 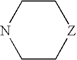 | — | N | C₂H₄OH |
| 17 | C₁₃ | O | 3 | CONR₂ | H | 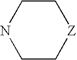 | — | O | — |

The ,post preferred amidoamine is Compound No. 4, which is known as N,N-Dimethyl-N′-tetradecanoyl-1,3-propylenediamine or N-[3-(Dimethylamino)propyl] tetradecanamide. This compound may also be referred to by means of CAS Number 45267-19-4.

Some of the amidoamines utilized in the present invention are available from commercial sources. For example, Compound No. 4 is available as MIRISTOCOR®, myristamidopropyl dimethylamine phosphate, from Hoffman-La Roche Inc., Nutley, N.J. (USA), and as Schercodine M from Scher Chemicals Inc., Clifton, N.J. (USA); Compound No. 5 is available as LEXAMINE® L-13, lauramidopropyl dimethylamine, from Inolex Chemical Company, Philadelphia, Pa. (USA); and Compound No. 1 is available as LEXAMINE® S-13, stearamidopropyl dimethylamine, also from Inolex Chemical Company.

The above-described amidoamines can be synthesized in accordance with known techniques, including those described in U.S. Pat. No. 5,573,726 (Dassanayake, et al.), the entire contents of which are hereby incorporated in the present specification by reference. Examples of general reaction schemes which may be utilized are provided below.

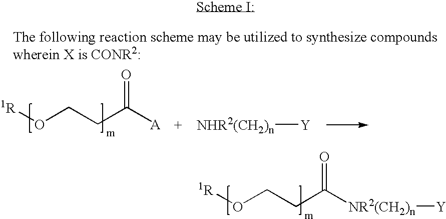

In the foregoing reaction scheme, A is a good leaving group, such as

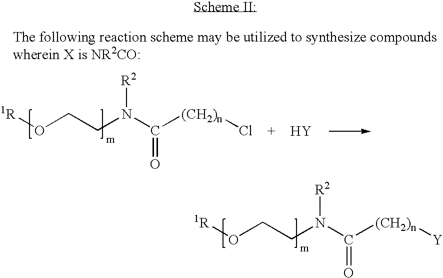

The following article may be referred to for further details concerning the Scheme I synthesis of the amidoamines of formula (I): Muzyczko, et al., “Fatty Amidoamine Derivatives: N,N-Dimethyl-N-(3-alkylamidopropyl)amines and Their Salts”, Journal of the American Oil Chemists' Society, volume 45, number 11, pages 720-725 (1968).

The compositions of the present invention contain one or more topically active antibiotics and one or more amidoamines of formula (I). The compositions may also contain other antimicrobial agents. For example, the compositions may contain cationic antiseptics. Examples of suitable cationic antiseptics include biguanides, such as chlorhexidine and PHMB, and quaternary-ammonium compounds, such as benzalkonium chloride and polyquaternium.

The amount of topically active antibiotic and the amount of amidoamines of formula (I) utilized in the compositions of the present invention will depend on the purpose of the use, e.g., the treatment of an active infection, the prophylactic treatment of tissues to prevent an active infection from developing, or the sterilization of tissues in conjunction with a medical procedure, such as a surgical procedure. The amounts utilized will also depend on the particular tissues being treated. For example, lower concentrations will typically be utilized to treat especially sensitive tissue, such as the eye, while somewhat higher concentrations may be utilized to treat less sensitive tissues, such as the nose. The concentrations determined to be necessary for the above-stated purposes can be functionally described as “an antiinfective amount”, “an antimicrobial effective amount” or variations thereof. The amounts of topically active antibiotics utilized will generally be in the range of from about 0.5 to about 10.0 weight/volume percent (w/v %), and the amounts to amidoamines utilized will generally be in the range of about 0.00001 to about 0.1 w/v %.

The above-described topically active antibiotics and amidoamines of formula (I) may be included in various types of pharmaceutical compositions, including solutions, suspensions, gels, ointments, creams, sprays and powders. The compositions may be aqueous or nonaqueous, but will generally be aqueous. As will be appreciated by those skilled in the art, the compositions may contain a wide variety of ingredients, such as tonicity agents (e.g., sodium chloride or mannitol), surfactants (e.g., polyoxyethylene/polyoxypropylene copolymers, such as Poloxamine™), viscosity adjusting agents (e.g., hydroxypropyl methyl cellulose and other cellulose derivatives) and buffering agents (e.g., borates, citrates, phosphates and carbonates). The present invention is not limited with respect to the types of pharmaceutical compositions in which the combination of one or more topically active antibiotics and one or more amidoamines of formula (I) may be utilized.

As will be appreciated by those skilled in the art, ophthalmic compositions intended for direct application to the eye will be formulated so as to have a pH and tonicity which are compatible with the eye. This will normally require a buffer to maintain the pH of the composition at or near physiologic pH (i.e., 7.4) and may require a tonicity agent to bring the osmolality of the composition near to 300 milliosmoles.

The following examples are presented to further illustrate methods of synthesizing the amidoamines of formula (I) and pharmaceutical compositions containing these compounds in combination with natamycin or other topically active antibiotics.

### EXAMPLE 1

Synthesis of N,N-Dimethyl-N′-Tetradecanoyl-1,3-Propylenediamine (Compound No. 4)

2.0 g. (0.0196 moles) of 3-dimethylaminopropylamine in 40 ml chloroform was added dropwise to an ice cold chloroform solution (50 ml) of myristoyl chloride (4.17 g., 0.0169 moles). After addition, the ice bath was removed and the solution was stirred for 2 hours. A 25 ml aqueous sodium bicarbonate solution was added and stirred for 30 minutes. The organic layer was then washed with 30 ml aqueous sodium bicarbonate/sodium chloride solution and dried with magnesium sulfate. The solution was concentrated in vacuo and the amide was recrystallized in ethyl acetate to yield 3.29 g. (0.0105 moles, 62.3%) of the subject compound.

¹H NMR (200 MHz, CDCL₃): δ 6.9 (s, 1H, NH), 3.3 (q, 3H, NHCH₂), 2.4 (t, 2H, NCH₂), 2.22 (s, 6H, NCH₃), 2.15 (t, 2H, COCH₂), 1.7-1.5 (m, 4H, COCH₂CH₂ and NHCH₂CH₂), 1.25 (s, 20H, COCH₂CH₂(CH₂), 0.88 (t, 3H, CH₃).

Elemental Analysis: Calculated for C₁₉H₄₀N₂O (312.52): C, 73.02; H, 12.90; N, 8.96. Found: C, 72.96; H, 12.92; N, 8.93.

### EXAMPLE 2

The following formulation is an example of an aqueous suspension of the present invention. This formulation is suitable for topical application as a drop or spray to the eye, ear, nose or throat.

| Ingredient | Amount (w/v %) |
|---|---|
| Natamycin | 5.0 |
| Compound No. 4 | 0.005 |
| Benzalkonuim Chloride | 0.02 |
| Glycerin | 0 to 2.5 |
| Mannitol | 0 to 2.5 |
| Hydroxypropyl Ethyl Cellulose | 0 to 2.5 |
| NaOH/HCl | q.s. pH 7.4 |
| Purified Water | q.s. 100 |

### EXAMPLE 3

The following formulation is an example of an aqueous gel of the present invention. This formulation is suitable for topical application to the eye, ear, nose or throat.

| Ingredient | Amount (w/v %) |
|---|---|
| Moxifloxacin | 0.1 to 1.0 |
| Natamycin | 5.0 |
| Compound No. 4 | 0.001 to 0.01 |
| Boric Acid | 0.3 |
| Xanthan Gum | 0.1 to 5.0 |
| Sodium Chloride | 0.64 |
| NaOH/HCl | q.s. pH 4 to 8 |
| Purified Water | q.s. 100 |

### EXAMPLE 4

The following formulation is an example of an ointment of the present invention. This formulation is suitable for topical application to the eye, ear, nose or throat.

| Ingredient | Amount (w/v %) |
|---|---|
| Natamycin | 5.0 |
| Compound No. 4 | 0.00 1 to 0.1 |
| White Petrolatum | 1-50 |
| Boric Acid | 0.3 |
| Mineral Oil | q.s. |

### EXAMPLE 5

The following formulation is an example of a powder of the present invention. This formulation is suitable for topical application to the eye, ear, nose or throat.

| Ingredient | Amount (weight %) |
|---|---|
| Natamycin | 5.0 |
| Compound No. 4 | 0.001 to 0.01 |
| Moxifloxacin | 0.1 to 1.0 |
| Boric Acid | q.s. |

What is claimed is:

1. A topical antimicrobial composition useful in the treatment and prevention of fungal infections of the eye, ear, nose and throat, comprising:
an antiinfective amount of a topically active antibiotic;
an antiinfective amount of an amidoamine of the formula: 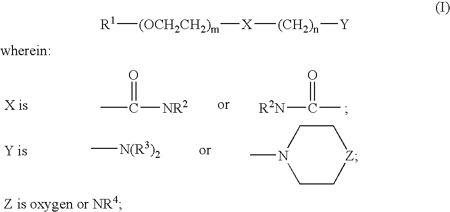
R¹ is C₆-C₁₈ saturated or unsaturated alkyl, alkylaryl, or alkoxyaryl;
m is zero to 16;
n is 2 to 16;
R², R³, and R⁴ are independently hydrogen, C₁-C₈ saturated or unsaturated alkyl or hydroxyalkyl, or a pharmaceutically acceptable salt thereof; and a pharmaceutically acceptable vehicle therefor.

2. A composition according to claim 1, wherein n is 2 to 4, and m is 0 to 5.

3. A composition according to claim 2, wherein R² is hydrogen or methyl, and R³ is methyl or ethyl.

4. A composition according to claim 1, wherein R¹ is heptadec-8-enyl, undecyl, undecenyl, dodecyl, tridecyl, tetradecyl, pentadecyl or heptadecyl, R² is hydrogen or methyl, R³ is methyl or ethyl, and R⁴ is hydrogen, methyl or hydroxyethyl.

5. A composition according to claim 1, wherein R¹ is tridecyl, m is 0, n is 3, Y is N(R³)₂ and R³ is methyl.

6. A composition according to claim 1, wherein the topically active antibiotic is selected from the group consisting of aminoglycoside antibiotics, quinolone antibiotics, and natamycin.

7. A composition according to claim 1, wherein the topically active antibiotic comprises natamycin.

8. A composition according to claim 7, wherein R¹ is tridecyl, m is 0, n is 3, y is N(³)₂ and R³ is methyl.

9. A method of treating or preventing fungal infections of the eye, ear or nose, which comprises applying a topical pharmaceutical composition to the affected tissues, said composition comprising:
an antiinfective amount of a topically active antibiotic;
an antiinfective amount of a compound of the following formula: 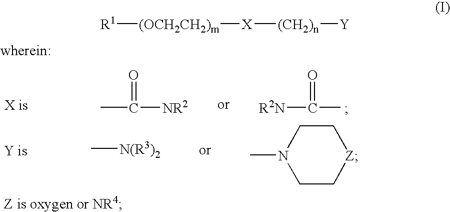
R¹ is C₆-C₁₈ saturated or unsaturated alkyl, alkylaryl, or alkoxyaryl;
m is zero to 16;
n is 2 to 16;
R², R³, and R⁴ are independently hydrogen, C₁-C₈ saturated or unsaturated alkyl or hydroxyalkyl, or a pharmaceutically acceptable salt thereof; and
a pharmaceutically acceptable vehicle therefor.

10. A method according to claim 9, wherein n is 2 to 4, and m is 0 to 5.

11. A method according to claim 10, wherein R² is hydrogen or methyl, and R³ is methyl or ethyl.

12. A method according to claim 9, wherein R¹ is heptadec-8-enyl, undecyl, undecenyl, dodecyl, tridecyl, tetradecyl, pentadecyl or heptadecyl, R² is hydrogen or methyl, R³ is methyl or ethyl, and R⁴ is hydrogen, methyl or hydroxyethyl.

13. A method according to claim 9, wherein R¹ is tridecyl, m is 0, n is 3, Y is N(³)₂ and R³ is methyl.

14. A method according to claim 9, wherein the composition further comprises 0.00005 to 0.01 w/v % of polyquaternium-1.

15. A method according to claim 9, wherein the topically active antibiotic is selected from the group consisting of aminoglycoside antibiotics, quinolone antibiotics, and natamycin.

16. A method according to claim 9, wherein the topically active antibiotic comprises natamycin.

17. A method according to claim 16, wherein R¹ is tridecyl, m is 0, n is 3, y is N(R³)₂ and R³ is methyl.
